(19) United States
(12) Patent Application Publication
(10) Pub. No.: US 2026/0124332 A1
(43) Pub. Date: May 7, 2026

(54) TEST DEVICE, STERILIZATION MONITORING SYSTEM AND METHOD

(71) Applicant: Solventum Intellectual Properties Company, Maplewood, MN (US)

(72) Inventors: G. Marco Bommarito, Stillwater, MN (US); Wensheng Xia, Woodbury, MN (US); Benjamin M. Wilke, Shoreview, MN (US); Naiyong Jing, St. Paul, MN (US)

(21) Appl. No.: 19/117,130

(22) PCT Filed: Sep. 13, 2023
(86) PCT No.: PCT/IB2023/059087

§ 371 Date: Mar. 31, 2025

Related U.S. Application Data
(60) Provisional application No. 63/378,561, filed on Oct. 6, 2022.

(51) Int. Cl. A61L 2/28; A61L 2/07; A61L 103/15; G01N 27/02

(52) U.S. Cl. CPC A61L 2/28; A61L 2/07; G01N 27/02; A61L 2103/15; A61L 2202/122; A61L 2202/14

(57) ABSTRACT

A test device for monitoring sterilization using a steam sterilant in a chamber is provided. The test device includes a test stack. The test stack includes an entrance layer including an entrance hole. The test stack further includes a sensor layer spaced apart from the entrance layer. The sensor layer includes a pair of electrodes disposed on the sensor layer. The test stack further includes a sensor coating disposed on a portion of the sensor layer and including an electrically active polymer. The test stack further includes a channel layer disposed between the entrance layer and the sensor layer. The channel layer includes an internal channel. The internal channel is configured to allow a flow of steam sterilant from the entrance hole to the sensor coating.

### TECHNICAL FIELD

The present disclosure relates generally to sterilization, and more particularly, relates to a test device for monitoring sterilization, a sterilization monitoring device including the test device, and a method for monitoring sterilization in a chamber.

### BACKGROUND

Sterilization of medical and hospital equipment may not be effective until a steam sterilant has been in contact with all surfaces of materials being sterilized in a proper combination of time, temperature, and steam quality. In steam sterilizers, such as pre-vacuum steam sterilizers and gravity displacement steam sterilizers, the process of sterilization is conducted in three main phases. In the first phase, air is removed, including air trapped within any porous materials being processed. The first phase is therefore an air removal phase. The second phase is a sterilizing stage, in which a load (i.e., the articles being sterilized) is subjected to steam under pressure for a recognized, predetermined combination of time and temperature to effect sterilization. The third phase is a drying phase in which condensation formed during the first two phases is removed by evacuating the chamber.

Any air that is not removed from the sterilizer during the air removal phase of the cycle or which leaks into the sterilizer during a sub atmospheric pressure stage due to, for example, faulty gaskets, valves or seals, may form air pockets within any porous materials present. Such air pockets may create a barrier to steam penetration, thereby preventing adequate sterilizing conditions being achieved for all surfaces of the load during the sterilizing phase. For example, these air pockets may prevent the steam from reaching interior layers of materials, such as hospital linens or fabrics. In some other examples, these air pockets may prevent the steam from penetrating hollow spaces of tubes, catheters, syringe needles, and the like. Further, non-condensable gas (generally air) present within the sterilizer is a poor sterilant and may decrease sterilization efficacy. A percentage of non-condensable gas in the steam should be less than or equal to 3.5% by volume. Therefore, the presence of air pockets and/or non-condensable gas may affect a steam quality of the steam sterilant. As a result, proper sterilization may not occur due to reduced steam quality. A few more factors that may affect steam quality include insufficient steam supply, water quality, degassing, design of the sterilizer chamber, etc.

### SUMMARY

In a first aspect, the present disclosure provides a test device for monitoring sterilization using a steam sterilant in a chamber. The test device includes a test stack defining a major plane and a perimeter. The test stack includes an entrance layer including an entrance hole extending through the entrance layer. The entrance hole is in fluidic connection with the chamber. The test stack further includes a sensor layer spaced apart from the entrance layer. The sensor layer includes a pair of electrodes disposed on the sensor layer. The test stack further includes a sensor coating disposed on a portion of the sensor layer and including an electrically active polymer. The sensor coating is spaced apart from the entrance hole at least along the major plane of the test stack. The sensor coating is electrically coupled to the pair of electrodes. The test stack further includes a channel layer disposed between the entrance layer and the sensor layer. The channel layer includes an internal channel defining a channel length along the major plane and a channel depth normal to the major plane. The internal channel is spaced apart from the perimeter of the test stack. The internal channel extends through the channel layer along the channel depth. The internal channel extends from the entrance hole to the sensor coating at least along the channel length, such that the internal channel fluidically connects the entrance hole with the sensor coating. The internal channel is configured to allow a flow of the steam sterilant from the entrance hole to the sensor coating. The sensor coating is configured to change an electrical impedance across the pair of electrodes upon contact of the steam sterilant with the sensor coating.

In a second aspect, the present disclosure provides a sterilization monitoring system including the test device of the first aspect. The sterilization monitoring system further includes a holder configured to at least partially and removably receive the test device therein.

In a third aspect, the present disclosure provides a sterilization system including the sterilization monitoring system of the second aspect. The sterilization system further includes a sterilizer including a chamber configured to receive the holder and the test device therein. The sterilizer is configured to perform a sterilization process on the test device using a steam sterilant within the chamber.

In a fourth aspect, the present disclosure provides a method for monitoring air removal in a chamber using the test device of first aspect. The method includes disposing the test device within the chamber. The method further includes performing a sterilization process on the test device using a steam sterilant. The method further includes removing the test device from the chamber. The method further includes at least partially inserting the test device within a reader for measuring the electrical impedance across the pair of electrodes.

In a fifth aspect, the present disclosure provides a test device for monitoring sterilization using a steam sterilant in a chamber. The test device includes a test stack defining a major plane and a perimeter. The test stack includes a top layer including a first major surface proximal to the chamber, a second major surface opposite to the first major surface, an entrance hole extending from the first major surface at least partially through the top layer and disposed in fluidic connection with the chamber, and an internal channel at least partially aligned with and disposed in fluidic connection with the entrance hole. The internal channel defines a channel length along the major plane and a channel depth normal to the major plane. The internal channel extends from the second major surface at least partially through the top layer along the channel depth. The internal channel is spaced apart from the perimeter of the test stack. The test stack further includes a sensor layer disposed adjacent to the second major surface of the top layer. The sensor layer includes a pair of electrodes disposed on the sensor layer. The test stack further includes a sensor coating disposed on a portion of the sensor layer and including an electrically active polymer. The internal channel of the top layer extends from the entrance hole to the sensor coating at least along the channel length, such that the internal channel fluidically connects the entrance hole with the sensor coating. The sensor coating is electrically coupled to the electrodes on the sensor layer.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 is a block diagram of a sterilization system, according to an embodiment of the present disclosure;

FIG. 2A is a perspective top view of a test device of the sterilization system of FIG. 1, according to an embodiment of the present disclosure;

FIG. 2B is a perspective bottom view of the test device of FIG. 2, according to an embodiment of the present disclosure;

FIG. 3A is a sectional side view of the test device of FIG. 2A comprising a test stack taken along a line A-A′ as shown in FIG. 2A, according to an embodiment of the present disclosure;

FIG. 3B is a sectional front view of the test device comprising the test stack taken along a line B-B′ as shown in FIG. 2A, according to an embodiment of the present disclosure;

FIG. 3C is a sectional front view of the test device comprising the test stack taken along a line C-C′ as shown in FIG. 2A, according to an embodiment of the present disclosure;

FIG. 4 is a top view of the test stack of FIG. 3A, with some layers not shown, according to an embodiment of the present disclosure;

FIG. 5 is a bottom view of the test stack of FIG. 3A, with some layers not shown, according to an embodiment of the present disclosure;

FIG. 6A is a bottom view of the test stack of FIG. 3A, with some layers not shown, according to another embodiment of the present disclosure;

FIG. 6B is a bottom view of the test stack of FIG. 3A, with some layers not shown, according to yet another embodiment of the present disclosure;

FIG. 7 schematically shows a reader of the sterilization system of FIG. 1, according to an embodiment of the present disclosure;

FIG. 8 is a sectional side view of a test device, according to another embodiment of the present disclosure;

FIG. 9 is a sectional side view of a test device, according to another embodiment of the present disclosure;

FIG. 10 is a sectional side view of a test device, according to another embodiment of the present disclosure;

FIGS. 11A to 11D are different views of a holder configured to at least partially and removably receive the test device of FIG. 2A, according to an embodiment of the present disclosure;

FIGS. 12A to 12D are different views of a holder configured to at least partially and removably receive the test device of FIG. 2A, according to another embodiment of the present disclosure;

FIGS. 13A and 13B are different views of the holder of FIG. 12A, with the test device of FIG. 2A at least partially received in the holder, according to an embodiment of the present disclosure;

FIG. 14 is a flowchart for a method for monitoring sterilization in a chamber using the test device of FIG. 2A, according to an embodiment of the present disclosure; and

FIG. 15 is a discrete plot of electrical impedance across a pair of electrodes of the test device of FIG. 2A in various test cycles; and

FIG. 16 is a graph illustrating a probability density function for logarithmic values of electrical impedance across the pair of electrodes of the test device of FIG. 2A.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

Steam sterilizers are widely used in medical centers and hospitals to sterilize medical equipment. Frequent testing or monitoring of steam quality may be essential to ensure a safe use of the medical equipment in a medical treatment. In other words, regular testing may have to be conducted to check effectiveness of air removal during air removal phase of the sterilization process, prior to subjecting the steam to a given load (i.e., medical equipment). One of the ways to monitor steam quality of the steam sterilant is Bowie-Dick test. In general, the Bowie-Dick test uses an indicator sheet and a test pack having stack of freshly laundered towels. In some cases, the indicator sheet is a chemical indicator sheet. In some cases, the indicator sheet is a bio indicator sheet. In some cases, the test pack used in the Bowie-Dick test includes a disposable test pack.

Although conventional technique of conducting the Bowie-Dick test by using the test pack is generally recognized as an adequate procedure for determining the steam quality of the steam sterilant or efficacy of the air removal stage of steam sterilization process, it may face some challenges. A uniform change in color of the indicator sheet indicates that all the air was removed and replaced by steam. In some cases, an operator may not accurately interpret a change in color of the indicator sheet, and this may further lead to erroneous classification of test results. Therefore, by using the test pack, the Bowie-Dick test may not always provide accurate test results due to possibility of human intervention errors while analyzing the test pack and/or the indicator sheet.

Further, to maintain a record/logbook of Bowie-Dick test results of one or more sterilizers, the operator may have to do a lot of scanning of the image of test packs, photocopying the test results, and manually recording the test results. This may be time consuming for the operator to manually maintain the logbook of the Bowie-Dick test results. As a result, throughput of a steam sterilizer may be reduced due to manual recording of the test results. Therefore, while using the test packs for conducting the Bowie-Dick tests, regularly updating the logbook of the Bowie-Dick test results may be difficult, erroneous, and time consuming. Moreover, for maintaining the logbook of the Bowie-Dick test results, a large quantity of paper may also be wasted on a regular basis.

The present disclosure relates to a test device for monitoring a steam quality of a steam sterilant in the chamber. The test device includes a test stack defining a major plane and a perimeter. The test stack includes an entrance layer including an entrance hole extending through the entrance layer. The entrance hole is in fluidic connection with the chamber. The test stack further includes a sensor layer spaced apart from the entrance layer. The sensor layer includes a pair of electrodes disposed on the sensor layer. The test stack further includes a sensor coating disposed on a portion of the sensor layer and including an electrically active polymer. The sensor coating is spaced apart from the entrance hole at least along the major plane of the test stack. The sensor coating is electrically coupled to the pair of electrodes. The test stack also includes a channel layer disposed between the entrance layer and the sensor layer. The channel layer includes an internal channel defining a channel length along the major plane and a channel depth normal to the major plane. The internal channel is spaced apart from the perimeter of the test stack. The internal channel extends through the channel layer along the channel depth. The internal channel extends from the entrance hole to the sensor coating at least along the channel length, such that the internal channel fluidically connects the entrance hole with the sensor coating. The internal channel is configured to allow a flow of the steam sterilant from the entrance hole to the sensor coating. The sensor coating is configured to change an electrical impedance across the pair of electrodes upon contact of the steam sterilant with the sensor coating. In an embodiment, the internal channel is at least partially non-linear along the channel length.

The present disclosure also provides a sterilization system including a sterilizer. The sterilizer includes a chamber configured to receive the test device. The sterilizer is configured to perform a sterilization process on the test device using the steam sterilant within the chamber.

For monitoring sterilization using the steam sterilant, the test device is placed within the chamber of the sterilizer and the sterilization process is initiated. As the internal channel fluidically connects the entrance hole with the sensor coating, and the entrance hole is in fluidic connection with the chamber, the chamber is in indirect fluidic connection with the sensor coating. In the presence of any non-condensable gas or air within the chamber, air may contact the sensor coating via the internal channel, and this may prevent the steam sterilant to make any contact with the sensor coating. Hence, in a real time sterilization process, in the presence of air, the steam sterilant may not reach hollow spaces and interior pockets of medical equipment subjected to sterilization. In the absence of the non-condensable gas or air within the chamber, the steam sterilant may be able to contact with the sensor coating via the internal channel. Hence, in the absence of air, the steam sterilant may reach hollow spaces and interior pockets of the medical equipment subjected to sterilization. In other words, for an acceptable quality of the steam sterilant, there should not be any presence of air within the chamber of the sterilizer.

As the internal channel can be at least partially non-linear along the channel length, the internal channel may offer a considerable channel resistance to flow of the steam sterilant through the internal channel. Particularly, the internal channel may provide the channel resistance that corresponds to a resistance provided by different routes and passages that the steam sterilant may have to follow to reach the hollow spaces and interior pockets of the medical equipment in a real time sterilization process. The channel resistance provided by the internal channel may therefore represent the resistance of various flow channels leading to hidden spaces of tubes, catheters, syringe needles, and the like. The channel resistance provided by the internal channel to the flow of the steam sterilant may depend on a shape and dimensions of the internal channel. Moreover, the shape and the dimensions of the internal channel may vary based on different application attributes.

Further, upon contact with the steam sterilant, the sensor coating is further configured to change the electrical impedance across the pair of electrodes beyond a predetermined threshold impedance. The predetermined threshold impedance may be selected based on various application attributes. Therefore, upon contact of the steam sterilant with the sensor coating, the electrical impedance across the pair of electrodes is beyond the predetermined threshold impedance. Further, in the presence of air in the internal channel, the steam sterilant may not contact with the sensor coating, and the electrical impedance across the pair of electrodes is below the predetermined threshold impedance.

The present disclosure further provides a sterilization monitoring system including the test device and a reader configured to at least partially receive the test device therein for measuring the electrical impedance across the pair of electrodes. The sterilization monitoring system is a part of the sterilization system of the present disclosure. Further, the entrance layer and the channel layer of the test device at least partially define a cutout disposed at the perimeter of the test stack. Each of the pair of electrodes at least partially extends into the cutout. The cutout is configured to at least partially receive one or more terminals of the reader therein for measuring the electrical impedance across the pair of electrodes. A magnitude of the electrical impedance across the pair of electrodes indicates the presence or absence of air in the sterilizer and the steam quality of the steam sterilant. The reader provides a pass result upon determining that the electrical impedance across the pair of electrodes is beyond the predetermined threshold impedance. Further, the reader provides a fail result upon determining that the electrical impedance across the pair of electrodes is below the predetermined threshold impedance. Therefore, the reader may provide an accurate pass or fail result of a steam sterilization process based on a comparison between the predetermined threshold impedance and the electrical impedance across the pair of electrodes.

In cases where the electrical impedance across the pair of electrodes is beyond the predetermined threshold impedance, the operator may also determine a quantitative relevancy of the pass result based on a magnitude of a difference between the electrical impedance and the predetermined threshold impedance. In cases where the electrical impedance across the pair of electrodes is not beyond the predetermined threshold impedance, the operator may also determine a quantitative relevancy of the fail result based on the magnitude of the difference between the electrical impedance and the predetermined threshold impedance.

Further, the test device is a built-in and a stand-alone unit which can be used with any sterilizer. In contrast to the conventional technique of monitoring sterilization by using the test pack and/or indicator sheets, and then manually interpreting the change in color of the indicator sheets, the sterilization monitoring system including the test device may require minimal human interpretation to determine the pass/fail result of the sterilization process. Therefore, the test device and the sterilization monitoring system of the present disclosure may provide an accurate classification of test results that could have been otherwise erroneous by reason of possible human intervention errors. Moreover, as the test device is being used here for monitoring the steam quality of the steam sterilant by measuring the electrical impedance across the pair of electrodes, the test device of the present disclosure may be called as an electronic testing unit or an electronic test card. In some cases, the sterilization monitoring system including the test device and the reader may also provide a digital pass/fail result of the steam quality of the steam sterilant.

In contrast to the conventional techniques for monitoring sterilization, the sterilization monitoring system of the present disclosure may eliminate a need for scanning of images of the test packs (indicator sheets), photocopying the test results, and manually recording the test results. Moreover, the sterilization monitoring system including the test device may eliminate the need to maintain a record/logbook of Bowie-Dick test results of one or more sterilizers. This may also reduce a possibility of misplacing the various test results of the steam quality of the steam sterilant. Therefore, an overall throughput of the sterilizer may be increased due to minimal manual recording and/or manual maintenance of the test results. The sterilization monitoring system may allow a faster and an easier testing process for the operator to accurately monitor the steam quality of the steam sterilant and/or validate proper air removal in the chamber of the sterilizer. Consequently, the disclosed sterilization monitoring system may increase an efficiency of the sterilizer and decrease a complexity of the process to monitor the steam quality of the steam sterilant. Moreover, the sterilization monitoring system may also save a large amount of paper that was otherwise wasted in the conventional techniques for monitoring sterilization.

The sterilization monitoring system of the present disclosure further includes a holder configured to at least partially and removably receive the test device therein. The holder is further configured to removably secure or hold the test device. For conducting a sterilization monitoring cycle, the holder and the test device at least partially received within the holder are placed in the chamber of the sterilizer. The holder may keep a position of the test device intact during a sterilization phase in the sterilizer. The holder is designed in such a way that it allows fluidic connection between the chamber of the sterilizer and the test device. Furthermore, the holder may have sufficient weight to removably secure the test device therein. The holder is made of a material, such that it is mechanically stable during sterilization cycles, and therefore, restrains mechanical motion of the test device during a sterilization cycle.

During the sterilization monitoring cycle, the holder may prevent any deformation or bulging of any of the layers of the test device. Moreover, the holder may also prevent delamination of the test device which may otherwise lead to erroneous test results of the steam quality of the steam sterilant. Therefore, the sterilization monitoring system including the holder and the test device received within the holder may improve accuracy of the test results.

As the holder is configured to removably secure the test device therein, a robustness of the test device may be reduced which can further lead to reduction in manufacturing cost of the test device. In some cases, the holder is manufactured as a single piece component comprising a plastic material. Further, the holder may be re-used several times for a number of sterilization monitoring cycles.

The sterilization monitoring system including the test device and the holder may also be used in other sterilization modalities, such as vaporized hydrogen peroxide sterilization. Moreover, the sterilization monitoring system may be used in different types of steam sterilizers that are already manufactured and are being currently used in the medical industry.

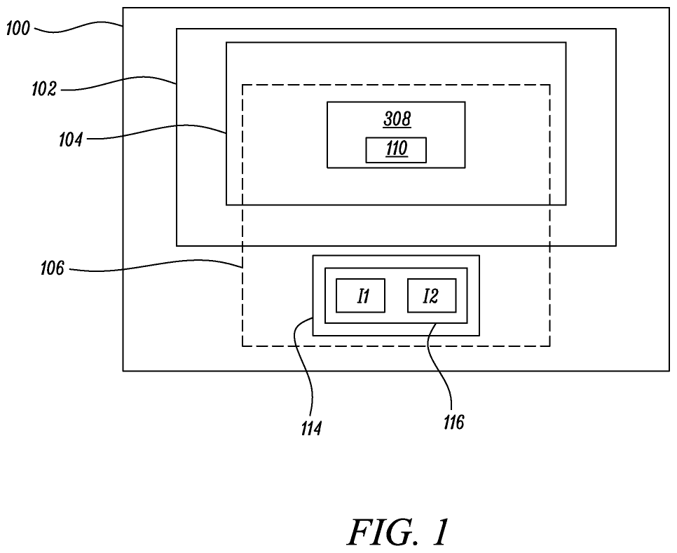

Referring now to Figures, FIG. 1 illustrates a block diagram of a sterilization system 100. The sterilization system 100 includes a sterilizer 102 including a chamber 104. The chamber 104 may have one or more environmental conditions. In some cases, the environmental condition may be related to conditions inside the chamber 104, and may include time, sterilant, temperature, pressure, or combinations thereof. In some embodiments, the chamber 104 may be made of various materials such as, but not limited to, steel, metal, polymer, or any other materials. The chamber 104 is configured to receive a steam sterilant therein. When steam is used as the steam sterilant, an object of a sterilization process is to bring steam at an appropriate temperature into contact with all surfaces of the articles being sterilized for an appropriate period of time.

The sterilization system 100 further includes a sterilization monitoring system 106. The sterilization monitoring system 106 includes a test device 110 for monitoring sterilization using the steam sterilant in the chamber 104. The sterilization monitoring system 106 further includes a holder 308 configured to at least partially and removably receive the test device 110 therein. The chamber 104 is configured to receive the holder 308 and the test device 110 therein. The sterilizer 102 is configured to perform the sterilization process on the test device 110 using the steam sterilant within the chamber 104. The holder 308 will be described later in the description.

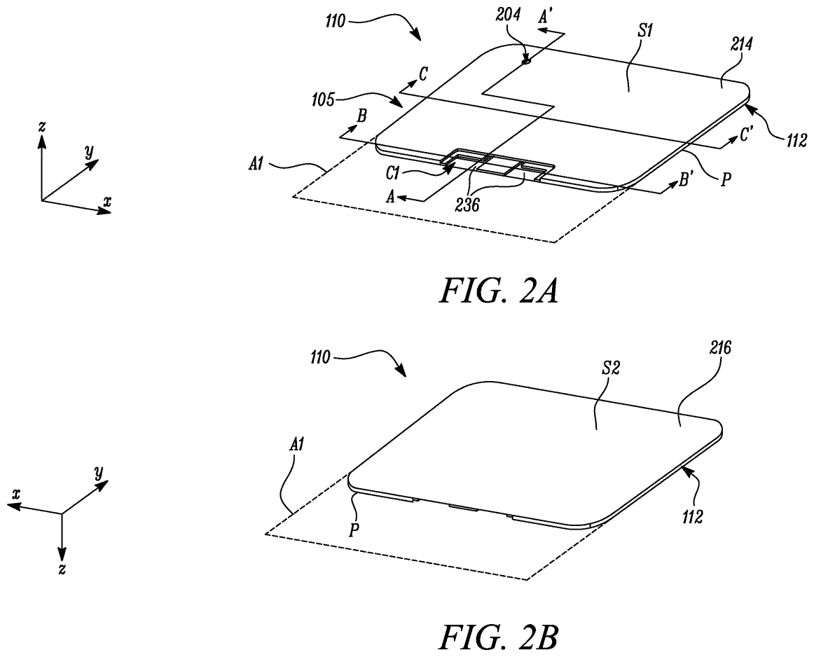

FIG. 2A is a perspective top view of the test device 110, according to an embodiment of the present disclosure. FIG. 2B is a perspective bottom view of the test device 110, according to an embodiment of the present disclosure. The test device 110 defines mutually orthogonal x, y, and z-axes. The test device 110 includes a test stack 112 defining a major plane A1 and a perimeter P. The x and y-axes are in-plane axes of the test stack 112, while the z-axis is a transverse axis disposed along a thickness of the test stack 112. In other words, the x and y-axes are disposed along the major plane A1 of the test stack 112, while the z-axis is perpendicular to the major plane A1 of the test stack 112. The major plane A1 therefore corresponds to the x-y plane.

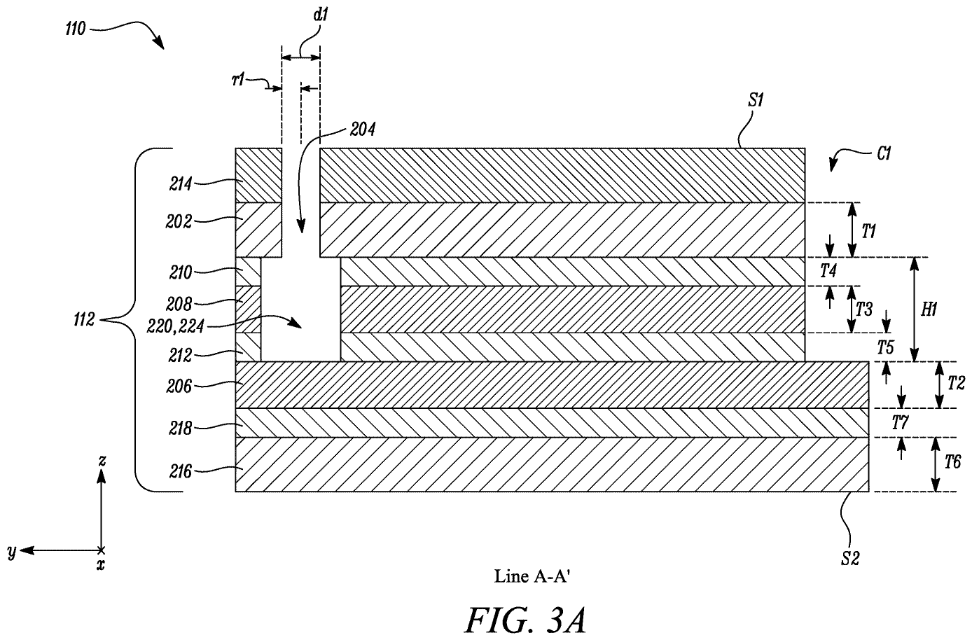

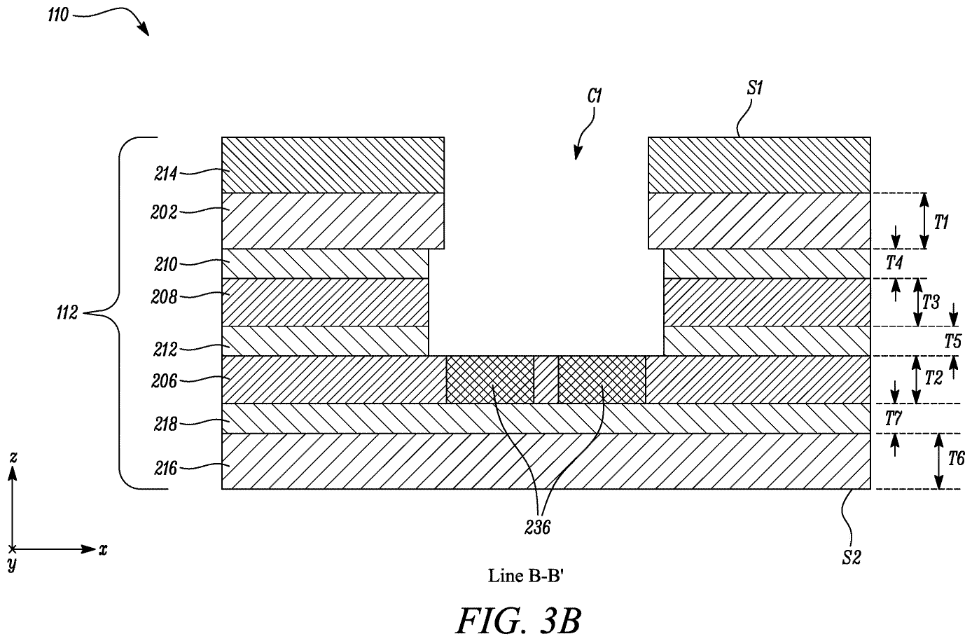

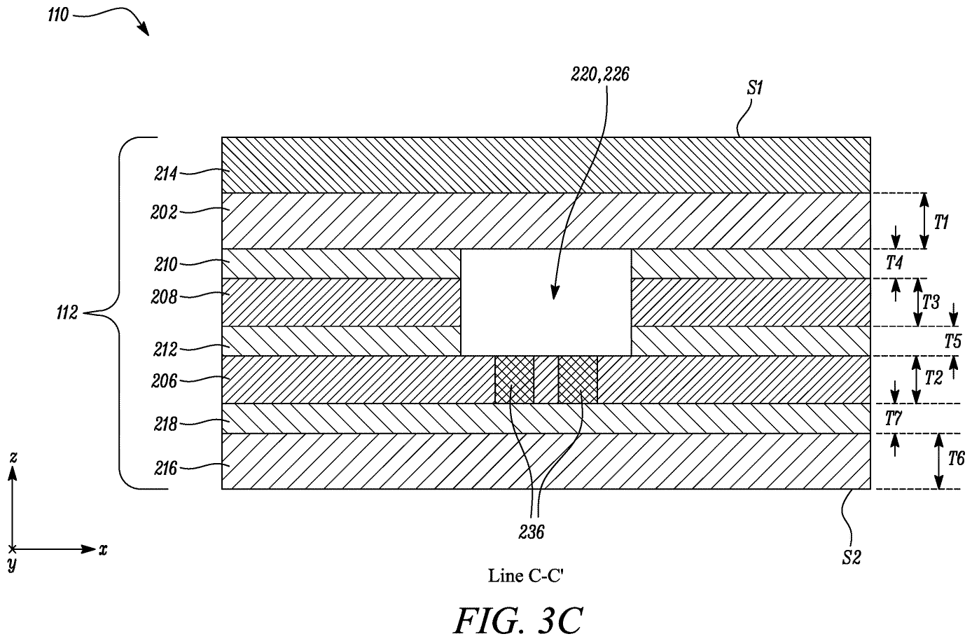

FIG. 3A is a sectional side view of the test device 110 comprising the test stack 112 taken along a line A-A′ as shown in FIG. 1, according to an embodiment of the present disclosure. The line A-A′ is zigzag shaped along the x-y plane. More particularly, FIG. 3A is a side sectional view of the test device 110 when viewed from a side 105 of the test device 110. FIG. 3B is a sectional front view of the test device 110 comprising the test stack 112 taken along a line B-B′ as shown in FIG. 2A, according to an embodiment of the present disclosure. FIG. 3C is a sectional front view of the test device 110 comprising the test stack 112 taken along a line C-C′ as shown in FIG. 2A, according to an embodiment of the present disclosure.

Referring to FIGS. 3A to 3C, the test stack 112 includes an entrance layer 202 including an entrance hole 204 extending through the entrance layer 202. In some embodiments, the entrance layer 202 includes polyethylene terephthalate (PET). Further, in some embodiments, the entrance layer 202 may be made of a metallic layer such as aluminum foil, a polymeric layer such as polyurethane or a polyester layer, without any limitations. The entrance layer 202 defines a thickness T1 along the z-axis. In some cases, the thickness T1 of the entrance layer is about 0.01 inches.

The entrance hole 204 is in fluidic connection with the chamber 104 (shown in FIG. 1). In the illustrated embodiment of FIG. 3A, the entrance hole 204 is circular and, therefore, has a diameter d1 and a radius r1 (d1/2). In some other embodiments, the entrance hole 204 may be of any other shape, such as square, triangular, rectangular, oval, elliptical, polygonal, or the like based on application attributes.

The test stack 112 further includes a sensor layer 206 spaced apart from the entrance layer 202. The sensor layer 206 defines a thickness T along the z-axis. In some embodiments, the thickness T of the sensor layer 206 is about 0.003 inches. In some embodiments, the thickness T2 of the sensor layer 206 is from about 10% to about 50% of the thickness T1 of the entrance layer 202.

The test stack 112 further includes a channel layer 208 disposed between the entrance layer 202 and the sensor layer 206. The channel layer 208 defines a thickness T3 along the z-axis. In some embodiments, the thickness T3 of the channel layer 208 is about 0.003 inches. In some embodiments, the thickness T3 of the channel layer 208 is from about 10% to about 50% of the thickness T1 of the entrance layer 202. In some embodiments, the channel layer 208 includes PET. Further, in some other embodiments, the channel layer 208 may be made of a metallic layer such as aluminum foil, a polymeric layer such as polyurethane or a polyester layer, without any limitations. Moreover, each of the entrance layer 202, the channel layer 208, and the sensor layer 206 is impermeable to the steam sterilant. Therefore, each of the entrance layer 202, the channel layer 208, and the sensor layer 206 may not allow a fluid (e.g., steam) to pass therethrough.

The test stack 112 further includes a first adhesive layer 210 disposed between the entrance layer 202 and the channel layer 208. The first adhesive layer 210 bonds the channel layer 208 to the entrance layer 202. In an example, the first adhesive layer 210 may include a very high bonding adhesive, such as a pressure sensitive adhesive, for example, but not limited to, silicone polyurea (SPU), acrylic, silicone, or rubber-based adhesive. In another example, the very high bonding adhesive may include structural adhesives, such as acrylic, cyanoacrylate, epoxy, polyurethane, or a mixture thereof. The first adhesive layer 210 defines a thickness T4 along the z-axis. In some cases, the thickness T4 of the first adhesive layer 210 is about 0.002 inches. In some embodiments, the thickness T4 of the first adhesive layer 210 is less than the thickness T3 of the channel layer 208.

The test stack 112 further includes a second adhesive layer 212 disposed between the channel layer 208 and the sensor layer 206. The second adhesive layer 212 bonds the channel layer 208 to the sensor layer 206. The second adhesive layer 212 defines a thickness T5 that is substantially equal to the thickness T4 of the first adhesive layer 210. Further, the second adhesive layer 212 may also include the very high bonding adhesive.

The test stack 112 further includes a graphics layer 214 disposed adjacent to the entrance layer 202 opposite to the channel layer 208. The graphics layer 214 at least partially forms an external surface S1 of the test stack 112. The entrance hole 204 further extends through the graphics layer 214.

In some embodiments, the graphics layer 214 may include a plurality of indicia (not shown), such as, but not limited to, letters, symbols, figures, pictures, logos, art, corporate messages, icons, etc., printed thereon. The plurality of indicia may be associated with and/or represent a business, a company or an organization or the like, or a product, service or the like, or both. In some examples, the graphics layer 214 may also include a code such as a Radio Frequency Identification (RFID) tag, a barcode, etc., printed thereon. Particularly, the plurality of indicia on the graphics layer 214 may display an information related to dates, serial numbers, product specifications, company logo, or usage markings of the test device 110.

The test stack 112 further includes a support layer 216 disposed adjacent to the sensor layer 206 opposite to the channel layer 208. The support layer 216 at least partially forms an external surface S2 of the test stack 112. The external surface S2 is disposed opposite to the external surface S1 formed by the graphics layer 214.

In some embodiments, the support layer 216 includes PET. In some other embodiments, the support layer 216 may be made of a metallic layer such as aluminum foil, a polymeric layer such as polyurethane or a polyester layer, without any limitations. The support layer 216 defines a thickness T6 along the z-axis. In some cases, the thickness T6 of the support layer 216 is about 0.01 inches. In some embodiments, the thickness T6 of the support layer 216 is substantially equal to the thickness T1 of the entrance layer 202. In some embodiments, the support layer 216 is impermeable to the steam sterilant.

In some embodiments, the entrance layer 202, the channel layer 208, the sensor layer 206, and the support layer 216 at least together form a laminated construction. The test stack 112 further includes a third adhesive layer 218 disposed between the sensor layer 206 and the support layer 216. The third adhesive layer 218 bonds the support layer 216 to the sensor layer 206. The third adhesive layer 218 defines a thickness T7 along the z-axis. In some embodiments, the third adhesive layer 218 may have a thickness of about 0.002 inches. In some embodiments, the third adhesive layer 218 may include a very high bonding adhesive. In an example, the thickness T4 of the first adhesive layer 210, the thickness T5 of the second adhesive layer 212, and the thickness 17 of the third adhesive layer 218 may be substantially equal to each other. In some embodiments, one or more layers of the test stack 112 may be transparent.

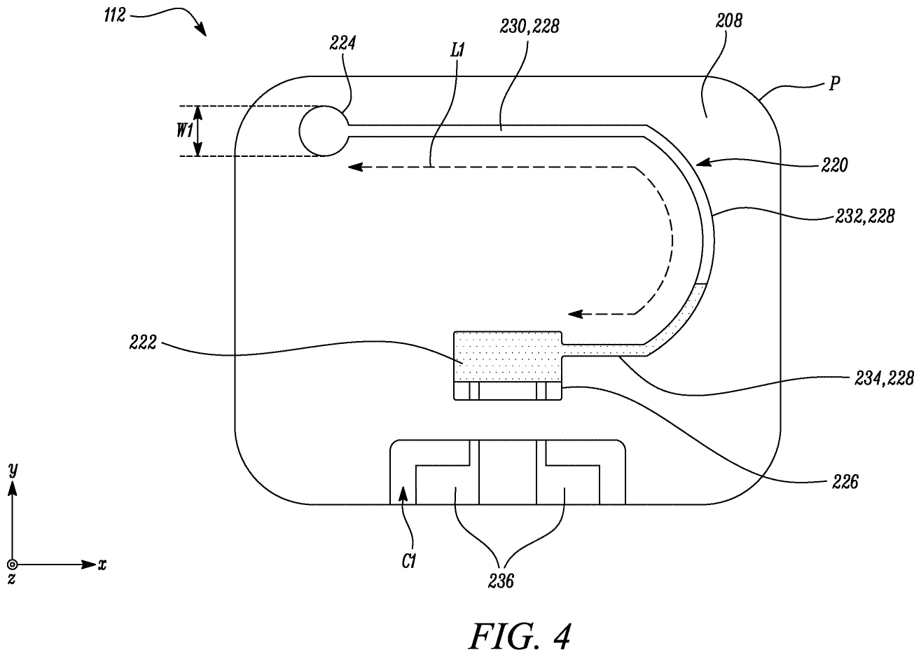

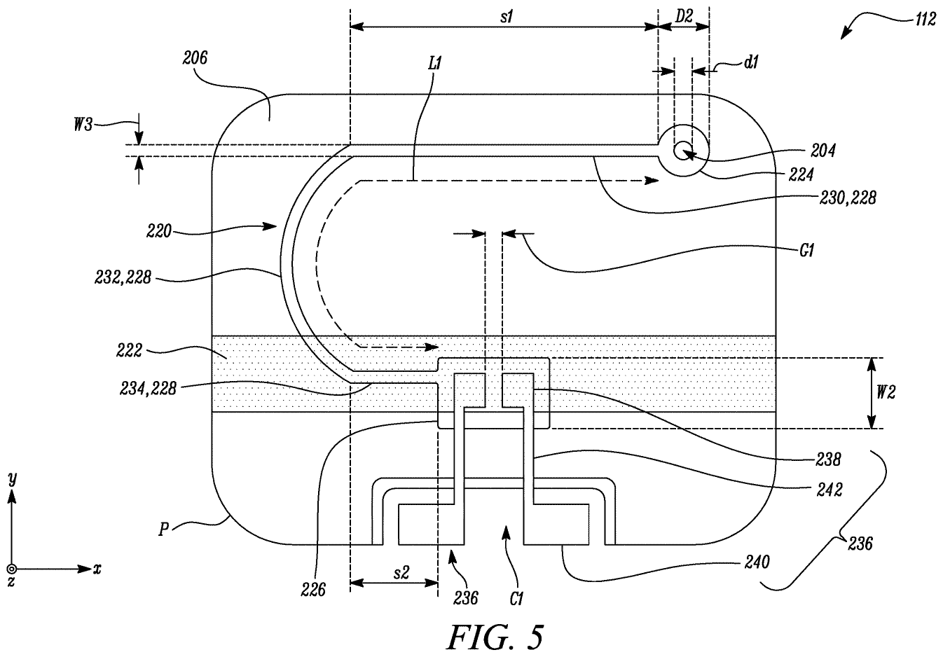

FIG. 4 illustrates a top view of the test stack 112 with some layers not shown, according to an embodiment of the present disclosure. Specifically, the graphics layer 214, the entrance layer 202, and the first adhesive layer 210 are not shown in the test stack 112 of FIG. 4. The channel layer 208 includes an internal channel 220 defining a channel length L1 (shown in FIG. 5) along the major plane A1 and a channel depth H1 (illustrated in FIG. 3A) normal to the major plane A1. The internal channel 220 is spaced apart from the perimeter P of the test stack 112. The internal channel 220 extends through the channel layer 208 along the channel depth H1. The internal channel 220 further extends through each of the first adhesive layer 210 and the second adhesive layer 212 along the channel depth H1. Specifically, the channel depth H1 extends through the thickness T4 of the first adhesive layer 210, the thickness T3 of the channel layer 208, and the thickness T5 of the second adhesive layer 212. In some embodiments, the channel depth H1 is from about 0.006 inches to about 0.008 inches.

FIG. 5 is a bottom view of the test stack 112, with some layers not shown, according to an embodiment of the present disclosure. Specifically, the support layer 216 and the third adhesive layer 218 are not shown in the test stack 112 of FIG. 5. The test stack 112 further includes a sensor coating 222 disposed on a portion of the sensor layer 206. The sensor coating 222 includes an electrically active polymer. The sensor coating 222 is spaced apart from the entrance hole 204 at least along the major plane A1 (shown in FIG. 2A) of the test stack 112. Therefore, the sensor coating 222 is spaced apart from the entrance hole 204 at least along the x-y plane of the test device 110.

In some embodiments, the electrically active polymer of the sensor coating 222 may include polyaniline (PANI), trans polyacetylene, poly (p-phenylene), poly (3-vinylperlene), polypyrrole, poly (2,5-bis (3-tetradecylthiophene-2-yl) thieno [3,2-b]thiophene), poly (2-(3-thienyyloxy) ethanesulfonate), polythiophene, or combinations thereof.

In some embodiments, the PANI may be in one of three oxidation states, i.e., leucoemeraldine, emeraldine (in a salt or base form), and per (nigraniline). The emeraldine may be less conductive in the base form and more conductive in the salt form. Further, the emeraldine salt may be converted into the leucoemeraldine salt or per (nigraniline) via a redox reaction to make the leucoemeraldine salt less conductive.

In some embodiments, the sensor coating 222 further includes tin. In some cases, the sensor coating 222 may include tin nanoparticles. In some other cases, the sensor coating 222 may include the PANI with blended nanoparticles of aluminum, copper, silver, gold, or combinations thereof.

With reference to FIGS. 4 and 5, the internal channel 220 extends from the entrance hole 204 to the sensor coating 222 at least along the channel length L1, such that the internal channel 220 fluidically connects the entrance hole 204 with the sensor coating 222. Therefore, the internal channel 220 is configured to allow a flow of the steam sterilant from the entrance hole 204 to the sensor coating 222. Moreover, the internal channel 220 is configured to allow a flow of non-condensable gas (e.g., air) from the entrance hole 204 to the sensor coating 222.

In some cases, the entrance hole 204 governs a flow rate of the non-condensable gas and/or the steam sterilant in and out of the internal channel 220. The flow rate is calculated according to Equation 1 provided below:

$$Flow Rate ∝ {( r 1 )}^{2} \qquad ( Equation 1 )$$

where, r1 is the radius of the entrance hole 204.

In some embodiments, the radius r1 of the entrance hole 204 may vary from about 0.05 mm to 8 mm.

In some embodiments, the internal channel 220 includes a first end portion 224 disposed in fluidic connection with the entrance hole 204. Therefore, the steam sterilant can flow from the chamber 104 (shown in FIG. 1) to the first end portion 224 of the internal channel 220 via the entrance hole 204. The first end portion 224 is at least partially aligned with the entrance hole 204. Specifically, the first end portion 224 is at least partially aligned with the entrance hole in the x-y plane. In the illustrated embodiment of FIGS. 4 and 5, the first end portion 224 is circular. In other embodiments, the first end portion 224 may be of any other desired shape, such as triangular, rectangular, oval, elliptical, polygonal, or the like shape, without any limitations. The first end portion 224 has a diameter D2. In some embodiments, the diameter D2 of the first end portion 224 is greater than the diameter d1 of the entrance hole 204 by a factor of at least 2. The first end portion 224 has a width W1 extending perpendicularly to the channel depth H1. In the illustrated embodiment of FIGS. 4 and 5, the width W1 of the first end portion 224 is same as the diameter D2 of the first end portion 224.

In some embodiments, the internal channel 220 further includes a second end portion 226 spaced apart from the first end portion 224 and disposed in fluidic connection with the sensor coating 222. Therefore, the steam sterilant can flow from the second end portion 226 to the sensor coating 222. The second end portion 226 is at least partially aligned with the sensor coating 222. Specifically, the second end portion 226 is at least partially aligned with the sensor coating 222 in the x-y plane. In the illustrated embodiment of FIGS. 4 and 5, the second end portion 226 is substantially rectangular. In other embodiments, the second end portion 226 may be of any other desired shape, such as triangular, rectangular, oval, elliptical, polygonal, or the like shape or may have rounded corners or rounded shape. The second end portion 226 has a width W2 extending perpendicularly to the channel depth H1.

In some embodiments, the internal channel 220 further includes a main portion 228 extending from the first end portion 224 to the second end portion 226 along the channel length L1. The main portion 228 is at least partially non-linear along the channel length L1. In other words, the internal channel 220 is at least partially non-linear along the channel length L1. The main portion 228 has a width W3. In some embodiments, the width W3 of the main portion 228 is less than the width W1, W2 of each of the first end portion 224 and the second end portion 226, respectively.

The main portion 228 includes a first linear section 230 extending from the first end portion 224, a curved section 232 extending from the first linear section 230, and a second linear section 234 extending from the curved section 232 to the second end portion 226. The first linear section 230 has a length s1. The second linear section 234 has a length s2. In the illustrated embodiment of FIGS. 4 and 5, the length s1 of the first linear section 230 is greater than the length s2 of the second linear section 234 by a factor of at least 2. Moreover, the first linear section 230, the curved section 232, and the second linear section 234 collectively define a substantial portion of the channel length L1 of the internal channel 220.

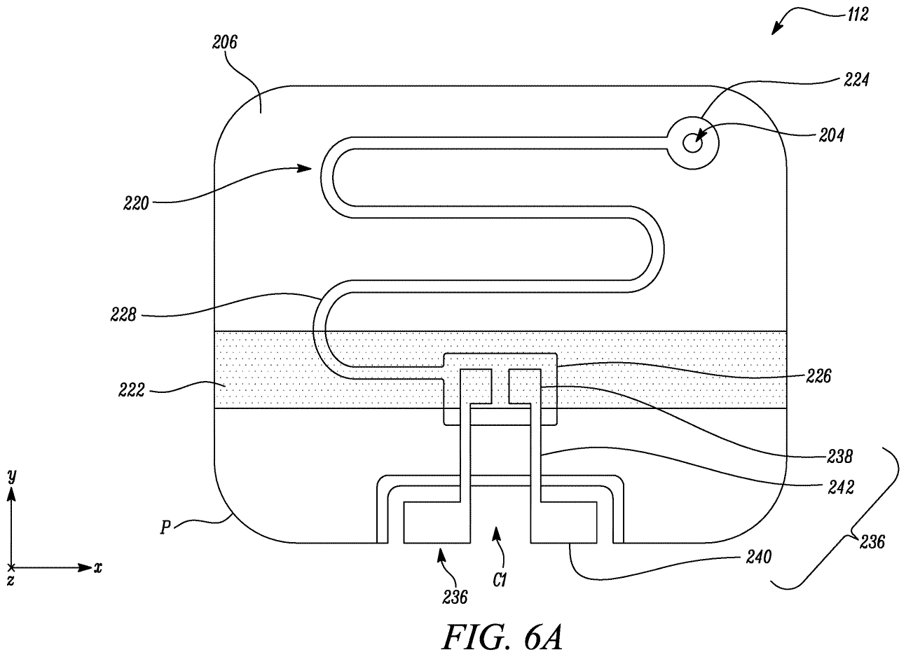

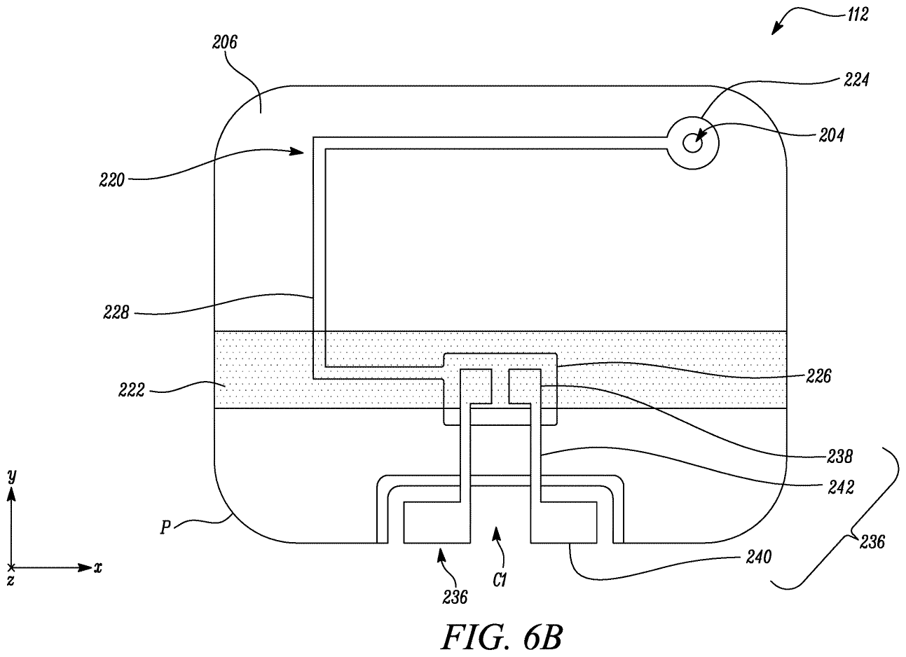

As shown in FIGS. 4 and 5, the main portion 228 includes two bends in total, i.e., one bend between the first linear section 230 and the curved section 232, and another bend between the curved section 232 and the second linear section 234. In some other embodiments, the main portion 228 may include more than two bends in total. FIG. 6A illustrates a bottom view of the test stack 112, according to another embodiment of the present disclosure. In this embodiment, the main portion 228 of the internal channel 220 has a serpentine shape having a plurality of bends. With an increase in the number of plurality of bends in the internal channel 220, a channel resistance provided by the internal channel 220 to the flow of the steam sterilant therethrough is also increased. The channel resistance may depend on a shape and dimensions of the internal channel 220. Moreover, the shape and the dimensions of the internal channel 220 may vary based on different application attributes.

A relationship between dimensions of the second end portion 226, the channel length L1, and a radius of the main portion 228 may be expressed as a diffusivity or a scaled diffusion length of the internal channel 220. The diffusivity is calculated according to Equation 2 provided below:

$${L}_{D} = \sqrt{\{ D + {( \frac{{r}_{h}^{3}}{8 μ} )}^{2} {( \frac{Δ P}{L} )}^{2} ( \frac{1}{48 D} ) \} t} \qquad ( Equation 2 )$$

where, L_{D} is the diffusivity of the internal channel 220;
D is either a diffusion constant of air during an air removal phase of a sterilization cycle or a diffusion constant of the steam sterilant during an exposure phase of the sterilization cycle;
rₕ is the radius of the main portion 228;
μ is either an air viscosity (during the air removal phase of the sterilization cycle) or steam sterilant viscosity (during the exposure phase of the sterilization cycle);
ΔP is a pressure difference across the internal channel 220;
L is an effective length of internal channel 220 (effective length of the internal channel 220 represents the channel length L1 plus an additional length proportional to a volume of the second end portion 226 and the radius rₕ of the main portion 228); and
t is time taken by air or the steam sterilant to flow through the internal channel 220.

In some embodiments, a value of the diffusivity L_{D} may range from about 0.02 cm to 60 cm.

FIG. 6B illustrates a bottom view of the test stack 112, according to yet another embodiment of the present disclosure. In this embodiment, the main portion 228 of the internal channel 220 has a rectangular shape with two bends. Specifically, the main portion 228 does not include any curved section. The two bends form the non-linear portions of the internal channel 220.

With reference to FIGS. 1 to 6B, for monitoring sterilization using the steam sterilant, the test device 110 is placed within the chamber 104 of the sterilizer 102 and the sterilization process is initiated. As the internal channel 220 fluidically connects the entrance hole 204 with the sensor coating 222, and the entrance hole 204 is in fluidic connection with the chamber 104, the chamber 104 is in indirect fluidic connection with the sensor coating 222. In some embodiments, any non-condensable gas or air present within the chamber 104 may contact the sensor coating 222 via the internal channel 220 and thus, prevent the steam sterilant to make any contact with the sensor coating 222. Therefore, in a real time sterilization process, in the presence of air, the steam sterilant may not reach hollow spaces and interior pockets of medical equipment subjected to sterilization. In the absence of the non-condensable gas or air within the chamber 104, the steam sterilant may be able to contact with the sensor coating 222 via the internal channel 220. Hence, in the absence of air, the steam sterilant may reach hollow spaces and interior pockets of the medical equipment subjected to sterilization. In other words, for an acceptable quality of the steam sterilant, there should not be any presence of air within the chamber 104 of the sterilizer 102.

As the internal channel 220 is at least partially non-linear along the channel length L1, the internal channel 220 may offer the considerable channel resistance to flow of the steam sterilant through the internal channel 220. Particularly, the at least partially non-linear internal channel 220 may provide the channel resistance that corresponds to a resistance provided by different routes and passages that the steam sterilant may have to follow to reach the hollow spaces and interior pockets of the medical equipment in a real time sterilization process. The channel resistance provided by the internal channel 220 may therefore represent the resistance of various flow channels leading to hidden spaces of tubes, catheters, syringe needles, and the like.

With reference to FIGS. 1 to 5, the sensor layer 206 includes a pair of electrodes 236 disposed on the sensor layer 206. Further, the sensor coating 222 is electrically coupled to the pair of electrodes 236. Each of the pair of electrodes 236 may include a conductive material. In some embodiments, each of the pair of electrodes 236 includes at least one of silver, carbon, and aluminum.

In some embodiments, at least a portion of each of the pair of electrodes 236 is disposed between the sensor coating 222 and the sensor layer 206, such that a gap G1 is defined between the pair of electrodes 236. The gap G1 is covered by the sensor coating 222. The second end portion 226 of the internal channel 220 at least surrounds the portion of each of the pair of electrodes 236 and the gap G1 between the pair of electrodes 236.

In some embodiments, the entrance layer 202 and the channel layer 208 at least partially define a cutout C1 disposed at the perimeter P of the test stack 112. Each of the pair of electrodes 236 at least partially extends into the cutout C1. Each of the pair of electrodes 236 includes a first rectangular portion 238 electrically coupled to the sensor coating 222, a second rectangular portion 240 disposed within the cutout C1, and a narrow elongate portion 242 connecting the first rectangular portion 238 to the second rectangular portion 240.

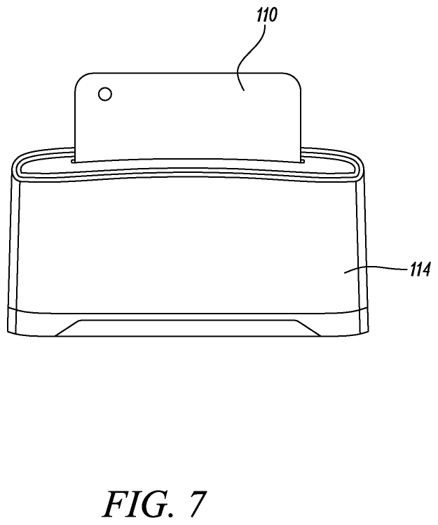

The sterilization monitoring system 106 further includes a reader 114 (shown in FIG. 1) configured to at least partially receive the test device 110 therein for measuring an electrical impedance I1 across the pair of electrodes 236. FIG. 7 schematically shows the reader 114, according to an embodiment of the present disclosure. Specifically, in FIG. 7, the test device 110 is received in the reader 114. A value of the electrical impedance I1 may be stored in a memory 116 of the reader 114.

Referring to FIGS. 1 to 7, the cutout C1 is configured to at least partially receive one or more terminals (not shown) of the reader 114 therein for measuring the electrical impedance I1 across the pair of electrodes 236. Further, the sensor coating 222 is configured to change the electrical impedance I1 across the pair of electrodes 236 upon contact of the steam sterilant with the sensor coating 222. In some embodiments, the sensor coating 222 is configured to change the electrical impedance I1 across the pair of electrodes 236 beyond a predetermined threshold impedance I2 (may be stored in the memory 116) upon contact with the steam sterilant.

Further, it should be noted that the electrically active polymer in the sensor coating 222 switches between one impedance state and another impedance state based on interaction with the steam sterilant. In some embodiments, as the pair of electrodes 236 may be coated with or formed from the conductive material such as silver or aluminum, the conductive material may directly react with the sensor coating 222 and convert emeraldine salt into leucoemeraldine salt to make the leucoemeraldine salt less conductive. The sensor coating 222 may therefore change from one impedance state to another impedance state based on the redox reaction of the electrically active polymer with the conductive material of the pair of electrodes 236 at the environmental condition of the chamber 104.

Moreover, in some embodiments, upon the appropriate exposure of the steam sterilant to the sensor coating 222, the pair of electrodes 236 may switch from being electrically shorted, i.e., a small impedance between the pair of electrodes 236 to being in an electrically open condition, i.e., a large impedance between the pair of electrodes 236.

While monitoring sterilization, the reader 114 provides a pass result upon determining that the electrical impedance I1 across the pair of electrodes 236 is beyond the predetermined threshold impedance I2. Further, the reader 114 provides a fail result upon determining that the electrical impedance I1 across the pair of electrodes 236 is below the predetermined threshold impedance I2. Therefore, the reader 114 may provide an accurate pass or fail result of a steam sterilization process based on a comparison between the predetermined threshold impedance I2 and the electrical impedance I1 across the pair of electrodes 236.

In cases where the electrical impedance I1 across the pair of electrodes 236 is beyond the predetermined threshold impedance I2, an operator may also determine a quantitative relevancy of the pass result based on a magnitude of a difference between the electrical impedance I1 and the predetermined threshold impedance I2. In cases where the electrical impedance I1 across the pair of electrodes 236 is not beyond the predetermined threshold impedance I2, the operator may also determine a quantitative relevancy of the fail result based on the magnitude of the difference between the electrical impedance I1 and the predetermined threshold impedance I2.

Further, the test device 110 is a built-in and a stand-alone unit which can be used with any sterilizer. In contrast to a conventional technique of monitoring sterilization by using test packs and/or indicator sheets, and then manually interpreting the change in color of the indicator sheets, the sterilization monitoring system 106 including the test device 110 may require minimal human interpretation to determine the pass/fail result of the sterilization process. Therefore, the test device 110 and the sterilization monitoring system 106 of the present disclosure may provide an accurate classification of test results that could have been otherwise erroneous by reason of possible human intervention errors. Moreover, as the test device 110 is being used here for monitoring steam quality of the steam sterilant by measuring the electrical impedance I1 across the pair of electrodes 236, the test device 110 of the present disclosure may be called as an electronic testing unit or an electronic test card. In some cases, the sterilization monitoring system 106 including the test device 110 and the reader 114 may also provide a digital pass/fail result of the steam quality of the steam sterilant.

In contrast to conventional techniques for monitoring sterilization, the sterilization monitoring system 106 of the present disclosure may eliminate a need for scanning of images of the test packs (indicator sheets), photocopying the test results, and manually recording the test results. Moreover, the sterilization monitoring system 106 including the test device 110 may eliminate the need to maintain a record/logbook of Bowie-Dick test results of one or more sterilizers. This may also reduce a possibility of misplacing the various test results of the steam quality of the steam sterilant. Therefore, an overall throughput of the sterilizer 102 may be increased due to minimal manual recording and/or manual maintenance of the test results. The sterilization monitoring system 106 may allow a faster and an easier testing process for the operator to accurately monitor the steam quality of the steam sterilant and/or validate proper air removal in the chamber 104 of the sterilizer 102. Consequently, the disclosed sterilization monitoring system 106 may increase an efficiency of the sterilizer 102 and decrease a complexity of the process to monitor the steam quality of the steam sterilant. Moreover, the sterilization monitoring system 106 may also save a large amount of paper that was otherwise wasted in the conventional techniques for monitoring sterilization.

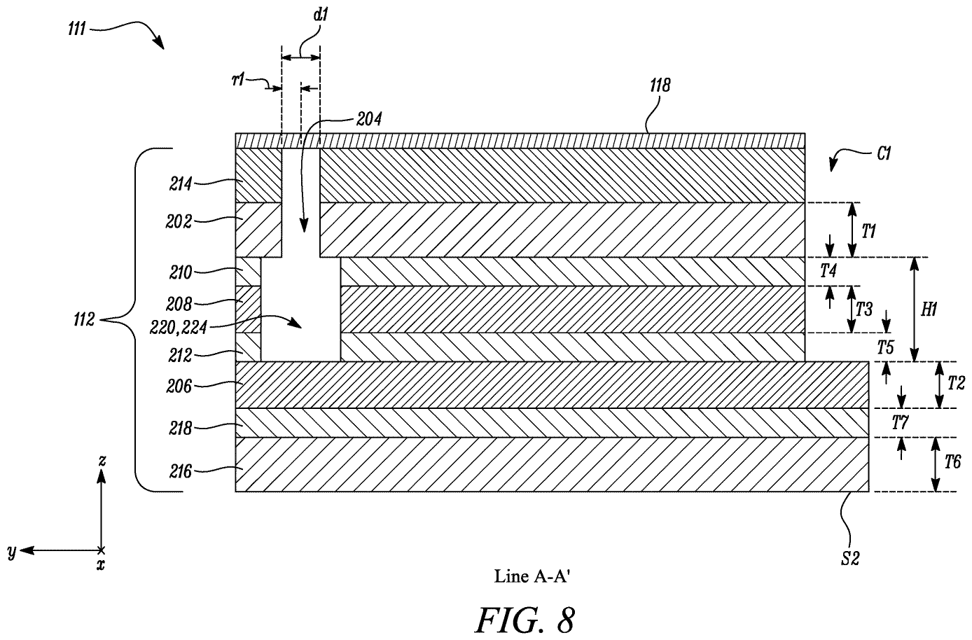

FIG. 8 is a sectional side view of a test device 111, according to another embodiment of the present disclosure. The sectional side view of the test device 111 is taken along the line A-A′ shown in FIG. 2A. The test device 111 is substantially similar to the test device 110 illustrated in FIG. 3A, with common components being referred to by the same reference numerals. However, the test device 111 further includes a porous film 118 disposed on the test stack 112 and covering the entrance hole 204. The porosity of the porous film 118 allows fluidic connection between the chamber 104 and the entrance hole 204. Specifically, the porous film 118 allows the steam sterilant to flow from the chamber 104 to the entrance hole 204, and subsequently to the sensor coating 222 via the internal channel 220. In some embodiments, the inclusion of the porous film 118 may increase an overall resistance provided by the test device 111 to the flow of the steam sterilant.

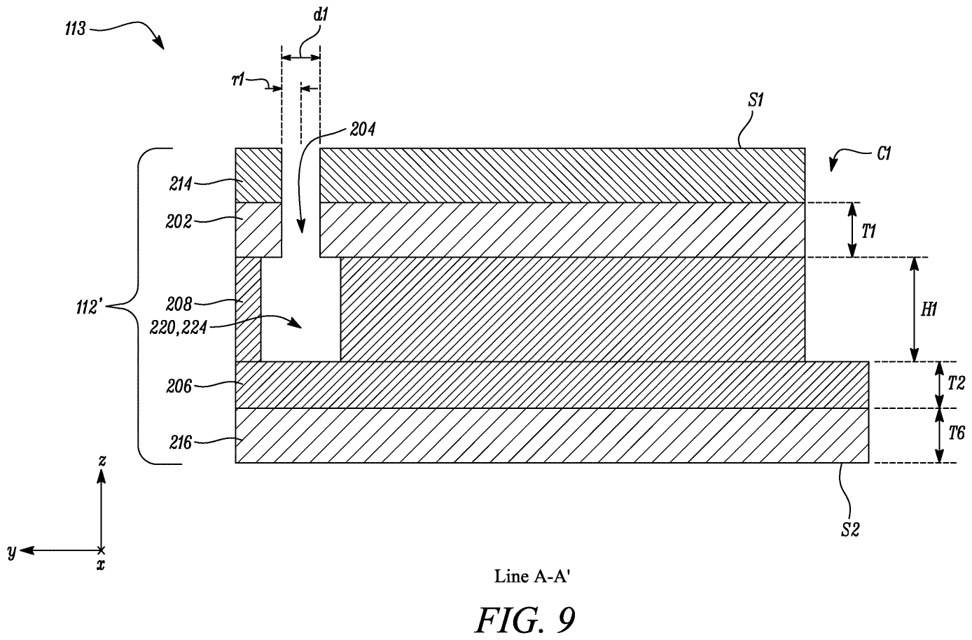

FIG. 9 is a sectional view of a test device 113, according to another embodiment of the present disclosure. The sectional side view of the test device 113 is taken along the line A-A′ shown in FIG. 2A. The test device 113 is substantially similar to the test device 110 illustrated in FIG. 3A, with common components being referred to by the same reference numerals. Further, the test device 113 includes a test stack 112′ substantially similar to the test stack 112 of the test device 110 illustrated in FIG. 3A, with common components being referred to by the same reference numerals. However, in the test stack 112′, there is no adhesive layer to bond the entrance layer 202 to the channel layer 208. Further, there is no adhesive layer to bond the channel layer 208 to the sensor layer 206. Moreover, there is no adhesive layer to bond the sensor layer 206 to the support layer 216. Therefore, the test stack 112′ is devoid of any separate adhesive layers. In the illustrated embodiment of FIG. 9, various adjacent layers of the test stack 112′ may be welded, or laminated, or ultrasonically bonded to each other. For example, the channel layer 208 may be ultrasonically bonded to the sensor layer 206. For example, the sensor layer 206 may be welded to the support layer 216.

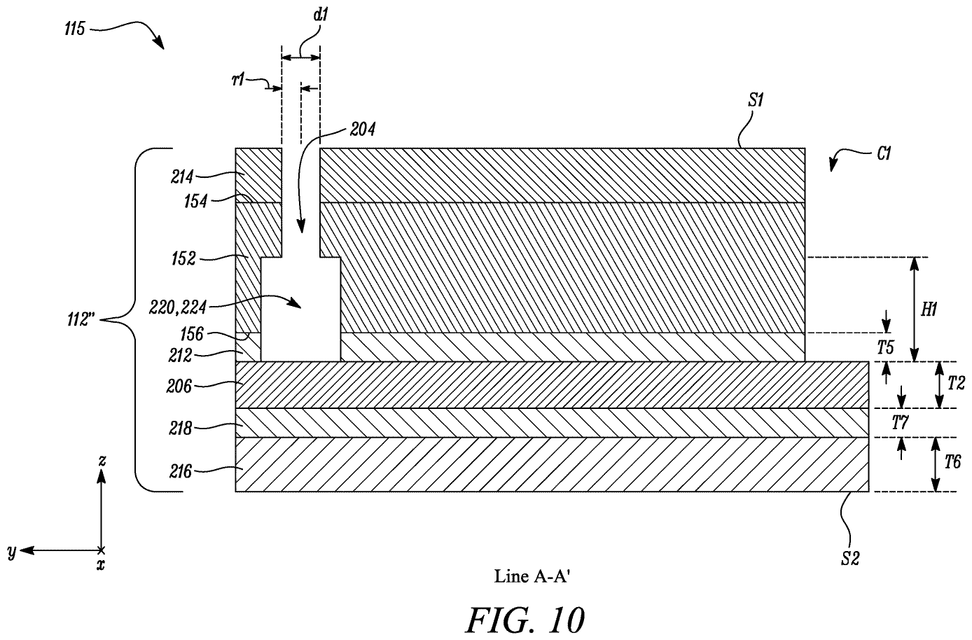

FIG. 10 is a sectional view of a test device 115, according to another embodiment of the present disclosure. The sectional side view of the test device 115 is taken along the line A-A′ shown in FIG. 2A. The test device 115 is substantially similar to the test device 110 illustrated in FIG. 3A, with common components being referred to by the same reference numerals. Further, the test device 115 includes a test stack 112″ substantially similar to the test stack 112 of the test device 110 illustrated in FIG. 3A, with common components being referred to by the same reference numerals.

However, the test stack 112″ includes a top layer 152 (instead of the entrance layer 202 and the channel layer 208 in the test stack 112 of FIG. 3A) including a first major surface 154 proximal to the chamber 104 and a second major surface 156 opposite to the first major surface 154. The top layer 152 further includes the entrance hole 204 extending from the first major surface 154 at least partially through the top layer 152 and disposed in fluidic connection with the chamber 104. The top layer 152 further includes the internal channel 220 at least partially aligned with and disposed in fluidic connection with the entrance hole 204. Specifically, the internal channel 220 is at least partially aligned with the entrance hole 204 in the x-y plane. The internal channel 220 extends from the second major surface 156 at least partially through the top layer 152 along the channel depth H1.

Further, the sensor layer 206 is disposed adjacent to the second major surface 156 of the top layer 152. Further, the sensor coating 222 is electrically coupled to the pair of electrodes 236 on the sensor layer 206 (shown in FIG. 4). Therefore, a main difference between the test device 115 of FIG. 10 and the test device 110 of FIG. 3A is that the test device 115 includes the top layer 152 which is a combination of the entrance layer 202 and the channel layer 208 illustrated in FIG. 3A.

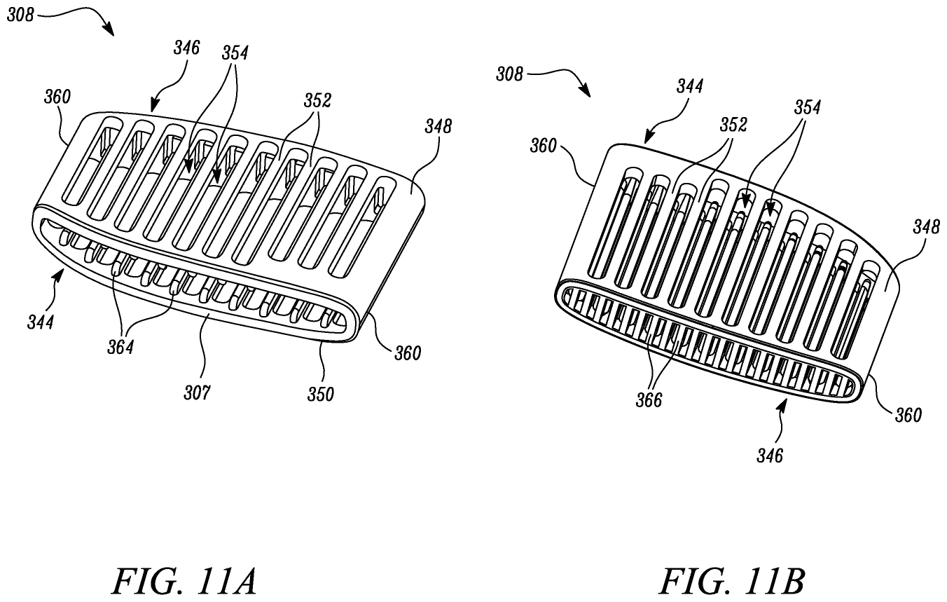

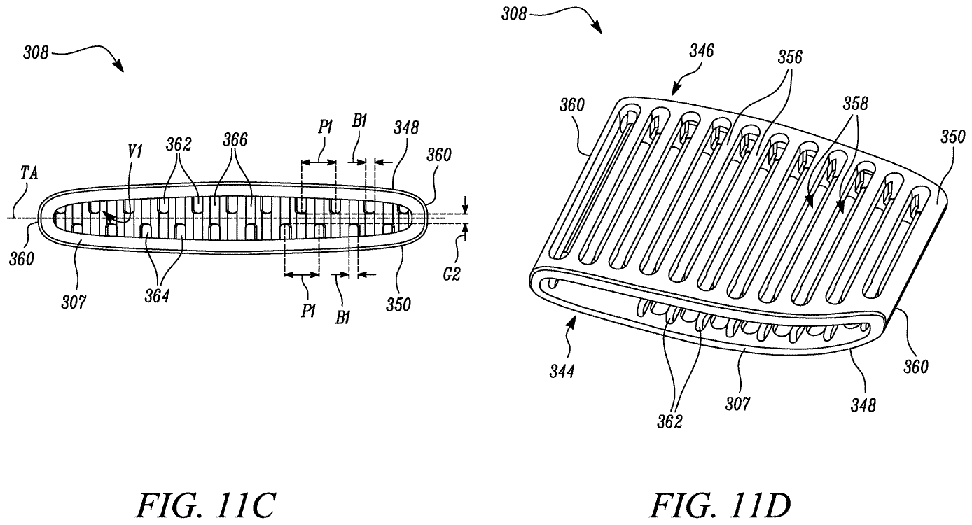

FIGS. 11A to 11D are different views of a holder 308 configured to at least partially and removably receive the test device 110 (shown in FIGS. 2A to 3C), according to an embodiment of the present disclosure.

The holder 308 includes a first open end 344 configured to at least partially receive the test device 110 therethrough. The holder 308 further includes a second open end 346 opposite to the first open end 344. The holder 308 includes a first portion 348 extending from the first open end 344 to the second open end 346. The holder 308 further includes a second portion 350 opposite to the first portion 348 and extending from the first open end 344 to the second open end 346. The holder 308 further includes a pair of lateral portions 360 disposed opposite to each other and connecting the first portion 348 to the second portion 350. The first portion 348, the second portion 350, and the pair of lateral portions 360 together define a volume V1 therebetween. The volume V1 extends from the first open end 344 to the second open end 346 and is configured to at least partially and removably receive the test device 110 therein. The holder 308 further defines a transverse axis TA extending between the pair of lateral portions 360.

The holder 308 further includes a plurality of first ribs 362 spaced apart from each other and extending from the first portion 348 towards the second portion 350. Each of the plurality of first ribs 362 at least partially extend between the first open end 344 and the second open end 346. The holder 308 further includes a plurality of second ribs 364 spaced apart from each other and extending from the second portion 350 towards the first portion 348. Each of the plurality of second ribs 364 at least partially extend between the first open end 344 and the second open end 346. The plurality of first ribs 362 and the plurality of second ribs 364 are configured to at least partially engage the test device 110 and removably secure the test device 110 therebetween. The holder 308 further includes a wall 307 surrounding each of the plurality of first ribs 362 and the plurality of second ribs 364. In some examples, the wall 307 may have a thickness of about 3 millimeters (mm) to 4 mm.

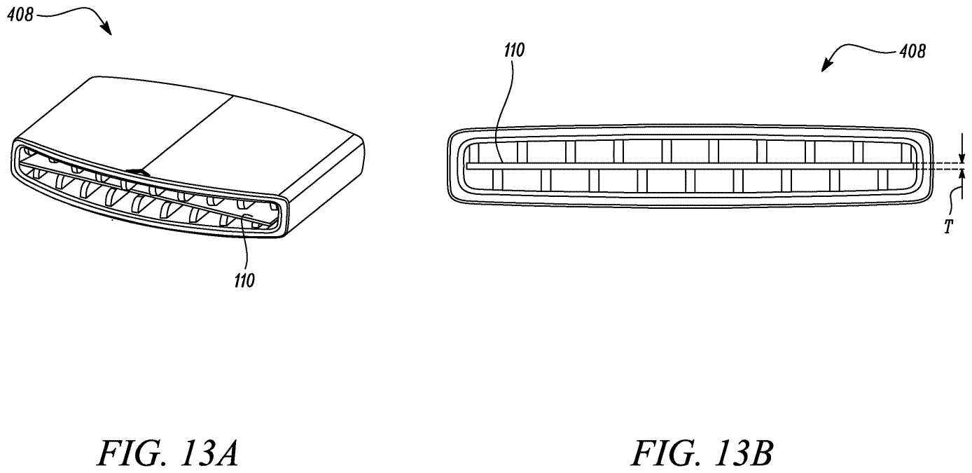

In some embodiments, the plurality of first ribs 362 and the plurality of second ribs 364 define a holder gap G2 therebetween. The holder gap G2 is a part of the volume V1 which at least partially and removably receives the test device 110 therein. In some embodiments, a height of the holder gap G2 is from about 0.03 inches to 0.075 inches. In some embodiments, the height of the holder gap G2 is from about 75% to 190% of a thickness T (shown in FIG. 13B) of the test device 110. The holder gap G2 allows the test device 110 to receive and expand during the sterilization monitoring cycles in the chamber 104.

In some embodiments, any two adjacent first ribs 362 from the plurality of first ribs 362 or any two adjacent second ribs 364 from the plurality of second ribs 364 define a pitch P1 therebetween. In some embodiments, the pitch P1 is from about 5 mm to 10 mm. Further, in some embodiments, a width B1 of each of the plurality of first ribs 362 and each of the plurality of second ribs 364 is from about 1 mm to 4 mm.

In the illustrated embodiment of FIGS. 11A to 11D, the plurality of first ribs 362 and the plurality of second ribs 364 are disposed in a staggered configuration relative to each other, such that at least one of the plurality of first ribs 362 is disposed between a pair of adjacent second ribs 364 from the plurality of second ribs 364 relative to the transverse axis TA extending between the pair of lateral portions 360. Further, at least one of the plurality of second ribs 364 is disposed between a pair of adjacent first ribs 362 from the plurality of first ribs 362 relative to the transverse axis TA.

In the illustrated embodiment of FIGS. 11A to 11D, the first portion 348 includes a plurality of first elongate members 352 spaced apart from each other and defining a plurality of first slots 354 therebetween. The second portion 350 includes a plurality of second elongate members 356 spaced apart from each other and defining a plurality of second slots 358 therebetween. Each of the plurality of first elongate members 352 and each of the plurality of second elongate members 356 extend between the first open end 344 and the second open end 346. The plurality of first slots 354 and/or the plurality of second slots 358 are configured to allow fluidic connection between the chamber 104 and the entrance hole 204 of the test device 110.

In the illustrated embodiment of FIGS. 11A to 11D, the plurality of first elongate members 352 and the plurality of second elongate members 356 are disposed in a staggered configuration relative to each other, such that at least one of the plurality of first elongate members 352 is disposed between a pair of adjacent second elongate members 356 from the plurality of second elongate members 356 relative to the transverse axis TA extending between the pair of lateral portions 360. Further, at least one of the plurality of second elongate members 356 is disposed between a pair of adjacent first elongate members 352 from the plurality of first elongate members 352 relative to the transverse axis TA.

In some embodiments, the holder 308 further includes a plurality of stop ribs 366 disposed at the second open end 346 and extending between the first portion 348 and the second portion 350. The plurality of stop ribs 366 are configured to at least partially engage the test device 110 thereby preventing the test device 110 from moving out of the holder 308 through the second open end 346.

In some embodiments, the holder 308 may be manufactured via a manufacturing process, for example, but not limited to, an additive manufacturing process, a molding process, or the like. In some cases, the holder 308 may be made of various materials, such as, but not limited to nylon, or any other materials. In some embodiments, the holder 308 is made of a material including aluminium, steel, machinable and 3D printable metal and metal alloys, polyphenylsulfone, polyethersulfone, polyetherimide, polyetherimidesulfone, and combination thereof.

In some embodiments, the holder 308 may be made by machining a metal via a turning process, a grinding process, a milling process, a drilling process or combination thereof. In some other embodiments, the holder 308 may be made by 3D printing methods such as stereolithography (SLA), selective laser sintering (SLS), fused deposition modeling (FDM), digital light process (DLP), multi jet fusion (MJF), polyjet, direct metal laser sintering (DMLS), electron beam melting etc., using heat stable 3D printable resin materials such as acrylonitrile butadiene styrene (ABS), thermoplastic polyurethane (TPU), composites and the like.

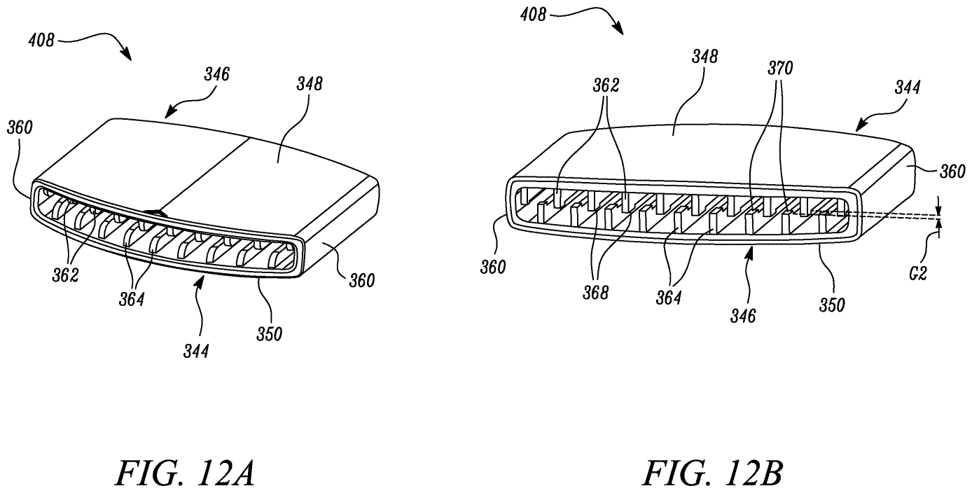

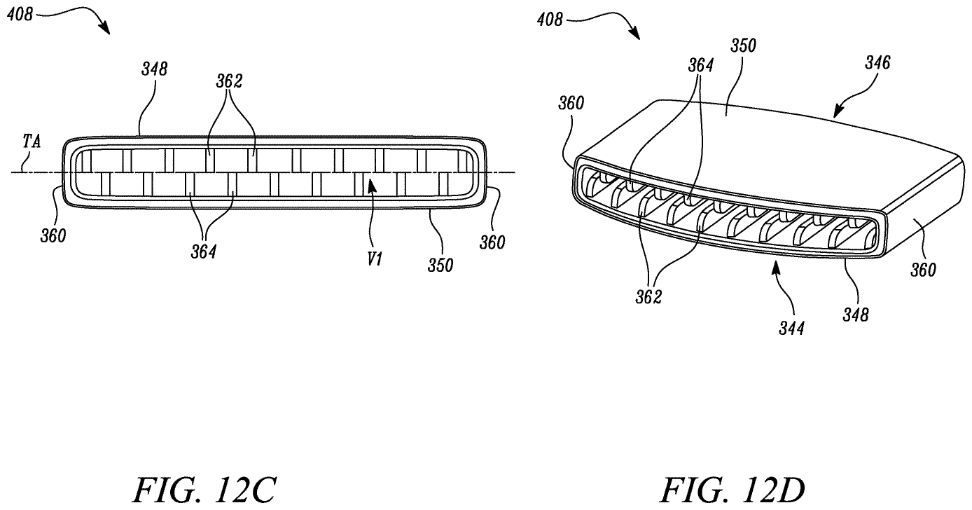

FIGS. 12A to 12D are different views of a holder 408 configured to at least partially and removably receive the test device 110 (shown in FIGS. 2A to 3C), according to another embodiment of the present disclosure. The holder 408 is functionally equivalent to the holder 308 illustrated in FIGS. 11A to 11D, with common components being referred to by the same reference numerals. However, in the holder 408, at least one of the first portion 348 and the second portion 350 has a substantially continuous planar shape devoid of openings. In the illustrated embodiment of FIGS. 12A to 12D, each of the first portion 348 and the second portion 350 has a substantially continuous planar shape devoid of openings. Therefore, in the holder 408, each of the first portion 348 and the second portion 350 does not include any elongate member (shown as the first elongate members 352 and the second elongate members 356 in FIGS. 11A to 11D).

Further, the holder 408 does not include any stop rib (shown as the stop ribs 366 in FIGS. 11A to 11D). However, the holder 408 includes a plurality of first stop projections 368 extending from the plurality of first ribs 362 at the second open end 346 and extending towards the second portion 350. The holder 408 further includes a plurality of second stop projections 370 extending from the plurality of second ribs 364 at the second open end 346 and extending towards the first portion 348. The plurality of first stop projections 368 and the plurality of second stop projections 370 are configured to at least partially engage the test device 110 thereby preventing the test device 110 from moving out of the holder 408 through the second open end 346.

The holder 408 is designed in such a way that it allows fluidic connection between the chamber 104 of the sterilizer 102 and the test device 110. In some cases, at least one of the first open end 344 and the second open end 346 allows fluidic connection between the chamber 104 and the test device 110.

FIGS. 13A and 13B are different views of the holder 408 with the test device 110 (shown in FIGS. 2A and 2B) at least partially received in the holder 408, according to an embodiment of the present disclosure. It should be noted that the holder 308 (shown in FIG. 11A) is also configured to least partially receive the test device 110 in a similar way as shown in FIGS. 13A and 13B.

Referring to FIGS. 1, and 11A to 13B, for conducting a sterilization monitoring cycle, the holder 408 (or any of the holders 308, 408) and the test device 110 at least partially received within the holder 408 are placed in the chamber 104 of the sterilizer 102. The holder 408 may keep a position of the test device 110 intact during a sterilization phase in the sterilizer 102. Therefore, the holder 408 may prevent any deformation or bulging of any of the layers of the test device 110 during the sterilization monitoring cycle. Moreover, the holder 408 may also prevent delamination of the test device 110 which may otherwise lead to erroneous test results of the steam quality of the steam sterilant. Hence, the sterilization monitoring system 106 including the holder 408 and the test device 110 received within the holder 408 may improve accuracy of the test results.

Further, the holder 408 may have sufficient weight to removably secure the test device 110 therein. The material of the holder 408 is chosen in such a way that it is mechanically stable during sterilization cycles, and therefore, restrains mechanical motion of the test device 110 during the sterilization cycle. In other words, the holder 408 is chosen in such a way that it may resist being forced apart due to deformation of the test device 110 caused by various test cycles for monitoring sterilization. In some embodiments, the holder 408 is made of a material having a minimum flexural modulus of 100 kpsi. In some embodiments, the holder 408 is made of a material having a flexural modulus in the range of 300 kpsi to 450 kpsi. In some cases, the flexural modulus of this particular range may help securing the test device 110 within the holder 408 and may prevent delamination due to various test cycles for monitoring sterilization.

In some embodiments, as the holder 408 is configured to removably secure the test device 110 therein, a robustness of the test device 110 may be reduced which can further lead to reduction in manufacturing cost of the test device 110. In some cases, the holder 408 is manufactured as a single piece component comprising a plastic material. Further, the holder 408 may be re-used several times for a number of sterilization monitoring cycles. A functional advantage of the holder 308 (shown in FIG. 11A) is substantially same as that of the holder 408.

The sterilization monitoring system 106 including the test device 110 and the holder 408 may also be used in other sterilization modalities, such as vaporized hydrogen peroxide sterilization. Moreover, the sterilization monitoring system 106 may be used in different types of steam sterilizers that are already manufactured and are being currently used in the medical industry.

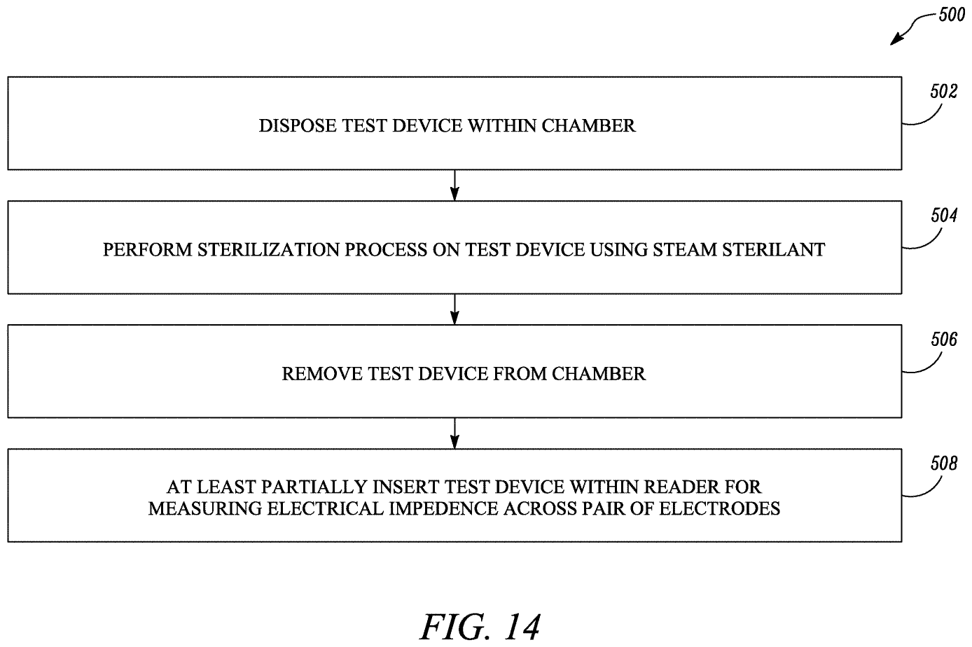

FIG. 14 illustrates a flow chart for a method 500 for monitoring air removal in the chamber 104 using the test device 110 (shown in FIG. 2A). With reference to FIGS. 1 to 10, at step 502, the method 500 includes disposing the test device 110 within the chamber 104. At step 504, the method 500 includes performing the sterilization process on the test device 110 using the steam sterilant. At step 506, the method 500 includes removing the test device 110 from the chamber 104. At step 508, the method 500 includes at least partially inserting the test device 110 within the reader 114 for measuring the electrical impedance I1 across the pair of electrodes 236.

### EXAMPLES

### Example 1

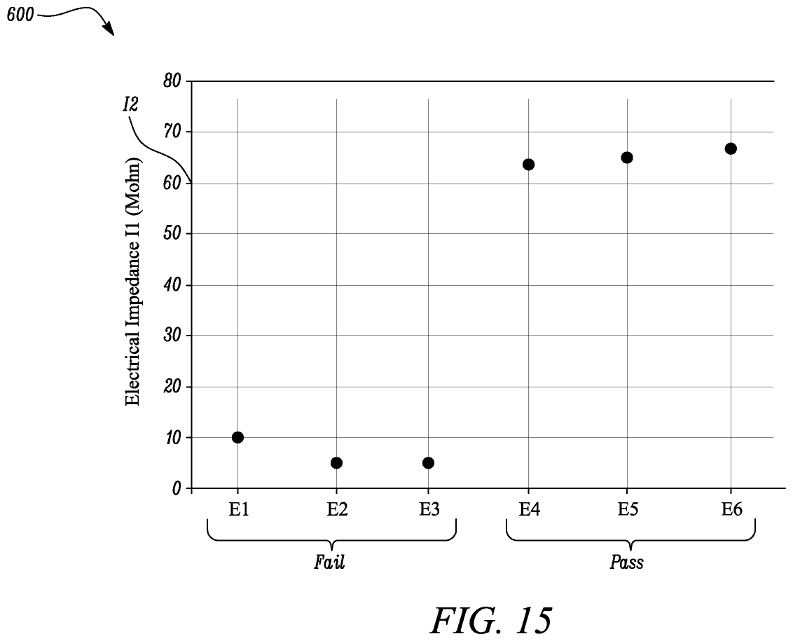

Various test cycles were performed to examine/validate the electrical impedance values across the pair of electrodes 236 (shown in FIG. 2A) of the test device 110. FIG. 15 is a discrete plot 600 of the electrical impedance I1 across the pair of electrodes 236 of the test device 110 in various test cycles. Specifically, in one of the experiments, six test cycles were performed. The electrical impedance I1 is depicted in Megaohms (Mohm) in the ordinate. Six test cycles are depicted in the abscissa.

A configuration of the test device 110 in first test cycle E1 was same as that of fourth test cycle E4. A configuration of the test device 110 in second test cycle E2 was same as that of fifth test cycle E5, and different than that of the first test cycle E1. A configuration of the test device 110 in third test cycle E3 was same as that of sixth test cycle E6, and different than that of the first test cycle E1 and the second test cycle E2. A configuration of the test device 110 was based on various parameters, such as material of the pair of electrodes 236, composition of the sensor coating 222 (shown in FIG. 5), thickness of various layers of the test device 110, the diameter d1 of the entrance hole 204, the channel length L1, the channel depth H1, and so on.

Moreover, for each of the first, second, and third test cycles E1, E2, E3, the Bowie-Dick test result was classified as a fail result. Further, for each of the fourth, fifth, and sixth test cycles E4, E5, E6, the Bowie-Dick test result was classified as a pass result. For each test cycle, a temperature inside the chamber 104 (shown in FIG. 1) of the sterilizer 102 was maintained at around 134 degrees Celsius. Further, for each test cycle, the predetermined threshold impedance I2 was estimated/set to about 60 Mohm.

Referring to the plot 600, it is apparent that the electrical impedance I1 in each of the first, second, and third test cycles E1, E2, E3 is well below (i.e., not beyond) the predetermined threshold impedance I2 (i.e., 60 Mohm). In other words, the electrical impedance I1 is below the predetermined threshold impedance I2 in all test cycles corresponding to fail Bowie-Dick test results. Further, it is apparent that the electrical impedance I1 in each of the fourth, fifth, and sixth test cycles E4, E5, E6 is beyond the predetermined threshold impedance I2 (i.e., 60 Mohm). In other words, the electrical impedance I1 is beyond the predetermined threshold impedance I2 in all test cycles corresponding to pass Bowie-Dick test results.

Therefore, from the plot 600, it can be concluded that all three configurations of the test device 110 provided accurate electrical impedance values corresponding to pass and/or fail Bowie-Dick test results. In other words, the electrical impedance I1 corresponding to the fail Bowie-Dick test result is clearly distinguishable from the electrical impedance I1 corresponding to the pass Bowie-Dick test result.

### Example 2

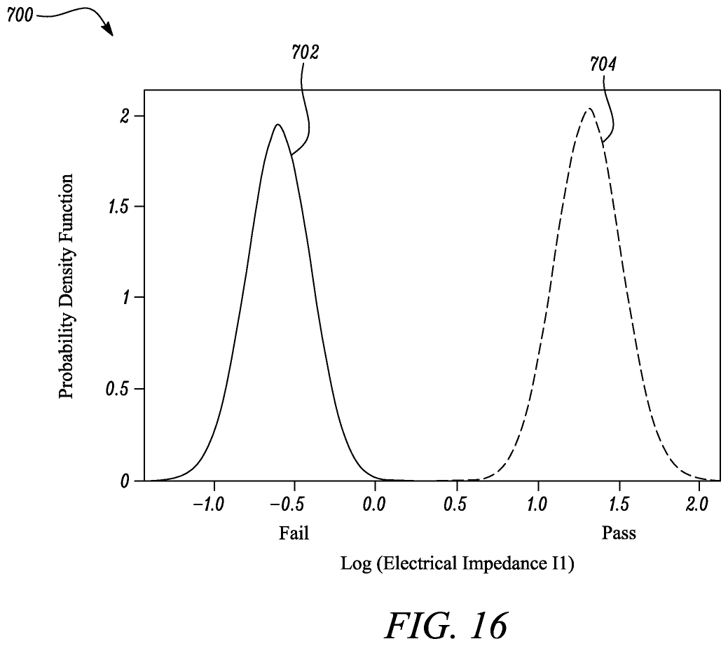

A test was performed to determine probability density functions for logarithmic values of the electrical impedance I1 across the pair of electrodes 236 of the test device 110 (shown in FIG. 2A). In this test, each of the pair of electrodes 236 included at least silver. The temperature inside the chamber 104 (shown in FIG. 1) of the sterilizer 102 was maintained at around 134 degrees Celsius. FIG. 16 is a graph 700 illustrating the probability density function for the logarithmic values of the electrical impedance I1 across the pair of electrodes 236 of the test device 110. The probability density function is depicted in the ordinate. The logarithmic values of the electrical impedance I1 are depicted in the abscissa. Specifically, in case of a fail Bowie-Dick test result, the probability density function for logarithmic values of the electrical impedance I1 is depicted by a curve 702. In case of a pass Bowie-Dick test result, the probability density function for logarithmic values of the electrical impedance I1 is depicted by a curve 704.

From the graph 700, it is apparent that the curves 702, 704 are clearly distinguishable from each other. In other words, the graph 700 depicts that the probability density function corresponding to the fail Bowie-Dick test is clearly distinguishable from the probability density function corresponding to the pass Bowie-Dick test. Such a segregation of the curves 702, 704 may eliminate erroneous test results while monitoring sterilization in the chamber 104. Moreover, from the curves 702, 704, an operator may also determine the quantitative relevancy of the pass/fail result of the Bowie-Dick test performed by the test device 110.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. A test device for monitoring sterilization using a steam sterilant in a chamber, the test device comprising:
a test stack defining a major plane and a perimeter, the test stack comprising:
an entrance layer comprising an entrance hole extending through the entrance layer, wherein the entrance hole is in fluidic connection with the chamber;
a sensor layer spaced apart from the entrance layer, wherein the sensor layer comprises a pair of electrodes disposed on the sensor layer;
a sensor coating disposed on a portion of the sensor layer and comprising an electrically active polymer, wherein the sensor coating is spaced apart from the entrance hole at least along the major plane of the test stack, wherein the sensor coating is electrically coupled to the pair of electrodes; and
a channel layer disposed between the entrance layer and the sensor layer, wherein the channel layer comprises an internal channel defining a channel length along the major plane and a channel depth normal to the major plane, wherein the internal channel is spaced apart from the perimeter of the test stack, wherein the internal channel extends through the channel layer along the channel depth, wherein the internal channel extends from the entrance hole to the sensor coating at least along the channel length, such that the internal channel fluidically connects the entrance hole with the sensor coating;
wherein the internal channel is configured to allow a flow of the steam sterilant from the entrance hole to the sensor coating, and wherein the sensor coating is configured to change an electrical impedance across the pair of electrodes upon contact of the steam sterilant with the sensor coating.

2. The test device of claim 1, wherein the internal channel is at least partially non-linear along the channel length.

3. The test device of claim 2, wherein the internal channel comprises a first end portion disposed in fluidic connection with the entrance hole, a second end portion spaced apart from the first end portion and disposed in fluidic connection with the sensor coating, and a main portion extending from the first end portion to the second end portion along the channel length, wherein the first end portion is at least partially aligned with the entrance hole, wherein the second end portion is at least partially aligned with the sensor coating, and wherein the main portion is at least partially non-linear along the channel length.

4. The test device of claim 3, wherein the first end portion is circular.

5. The test device of claim 4, wherein the entrance hole is circular, and wherein a diameter of the first end portion is greater than a diameter of the entrance hole by a factor of at least 2.

6. The test device of claim 3, wherein the second end portion is substantially rectangular.

7. The test device of claim 3, wherein the main portion comprises a first linear section extending from the first end portion, a curved section extending from the first linear section, and a second linear section extending from the curved section to the second end portion.

8. The test device of claim 7, wherein a length of the first linear section is greater than a length of the second linear section by a factor of at least 2.

9. The test device of claim 3, wherein the main portion has a serpentine shape having a plurality of bends.

10. The test device of claim 3, wherein a width of the main portion is less than a width of each of the first end portion and the second end portion.

11. The test device of claim 3, wherein at least a portion of each of the pair of electrodes is disposed between the sensor coating and the sensor layer, such that a gap is defined between the pair of electrodes, wherein the gap is covered by the sensor coating, and wherein the second end portion at least surrounds the portion of each of the pair of electrodes and the gap between the pair of electrodes.

12. The test device of claim 1, wherein the entrance layer and the channel layer at least partially define a cutout disposed at the perimeter of the test stack, wherein each of the pair of electrodes at least partially extends into the cutout, and wherein the cutout is configured to at least partially receive one or more terminals of a reader therein for measuring the electrical impedance across the pair of electrodes.

13. The test device of claim 12, wherein each of the pair of electrodes comprises a first rectangular portion electrically coupled to the sensor coating, a second rectangular portion disposed within the cutout, and a narrow elongate portion connecting the first rectangular portion to the second rectangular portion.

14. (canceled)

15. The test device of claim 1, wherein the test stack further comprises:
a first adhesive layer disposed between the entrance layer and the channel layer, the first adhesive layer bonding the channel layer to the entrance layer; and
a second adhesive layer disposed between the channel layer and the sensor layer, the second adhesive layer bonding the channel layer to the sensor layer;
wherein the internal channel further extends through each of the first adhesive layer and the second adhesive layer along the channel depth.

16.-22. (canceled)

23. The test device of claim 1, wherein the test stack further comprises a support layer disposed adjacent to the sensor layer opposite to the channel layer, wherein the support layer at least partially forms an external surface of the test stack.

24.-34. (canceled)

35. A sterilization monitoring system comprising;
the test device of claim 1; and
a holder configured to at least partially and removably receive the test device therein.

36. The sterilization monitoring system of claim 35, wherein the holder comprises:
a first open end configured to at least partially receive the test device therethrough;
a second open end opposite to the first open end;
a first portion extending from the first open end to the second open end;
a second portion opposite to the first portion and extending from the first open end to the second open end;
a pair of lateral portions disposed opposite to each other and connecting the first portion to the second portion, wherein the first portion, the second portion, and the pair of lateral portions together define a volume therebetween, wherein the volume extends from the first open end to the second open end and is configured to at least partially and removably receive the test device therein;
a plurality of first ribs spaced apart from each other and extending from the first portion towards the second portion, wherein each of the plurality of first ribs at least partially extend between the first open end and the second open end; and
a plurality of second ribs spaced apart from each other and extending from the second portion towards the first portion, wherein each of the plurality of second ribs at least partially extend between the first open end and the second open end;
wherein the plurality of first ribs and the plurality of second ribs are configured to at least partially engage the test device and removably secure the test device therebetween.

37.-50. (canceled)

51. A sterilization system comprising;
the sterilization monitoring system of claim 35; and
a sterilizer comprising a chamber configured to receive the holder and the test device therein, wherein the sterilizer is configured to perform a sterilization process on the test device using a steam sterilant within the chamber.

52. (canceled)

53. A test device for monitoring sterilization using a steam sterilant in a chamber, the test device comprising:
a test stack defining a major plane and a perimeter, the test stack comprising:
a top layer comprising a first major surface proximal to the chamber, a second major surface opposite to the first major surface, an entrance hole extending from the first major surface at least partially through the top layer and disposed in fluidic connection with the chamber, and an internal channel at least partially aligned with and disposed in fluidic connection with the entrance hole, the internal channel defining a channel length along the major plane and a channel depth normal to the major plane, wherein the internal channel extends from the second major surface at least partially through the top layer along the channel depth, and wherein the internal channel is spaced apart from the perimeter of the test stack;
a sensor layer disposed adjacent to the second major surface of the top layer, wherein the sensor layer comprises a pair of electrodes disposed on the sensor layer; and
a sensor coating disposed on a portion of the sensor layer and comprising an electrically active polymer, wherein the internal channel of the top layer extends from the entrance hole to the sensor coating at least along the channel length, such that the internal channel fluidically connects the entrance hole with the sensor coating, and wherein the sensor coating is electrically coupled to the pair of electrodes on the sensor layer.

54. The test device of claim 53, wherein the internal channel is configured to allow a flow of the steam sterilant from the entrance hole to the sensor coating, and wherein the sensor coating is configured to change an electrical impedance across the pair of electrodes upon contact of the steam sterilant with the sensor coating.
